(19) United States
(12) Patent Application Publication
(10) Pub. No.: US 2025/0161246 A1
(43) Pub. Date: May 22, 2025

(54) NOVEL COMPOSITIONS FOR THE MANAGEMENT OF HYPONATREMIA SUITABLE FOR PACKAGING IN MULTI-USE CONTAINER

(71) Applicant: NEPHCENTRIC, LLC, Phoenix, AZ (US)

(72) Inventors: Lee Souders, Albuquerque, NM (US); Bhupinder Singh, Phoenix, AZ (US)

(21) Appl. No.: 18/943,006

(22) Filed: Nov. 11, 2024

Related U.S. Application Data
(60) Provisional application No. 63/597,915, filed on Nov. 10, 2023.

(51) Int. Cl. A61K 31/17; A61K 9/00; A61K 33/06

(52) U.S. Cl. CPC A61K 31/17; A61K 9/0056; A61K 33/06

(57) ABSTRACT

The present invention involves novel urea compositions for oral administration, that are useful for treatment or management of hyponatremia that can be packaged in multi-use containers.

### FIELD OF THE INVENTION

This invention relates to novel urea compositions for oral administration that are useful for treating or managing hyponatremia.

### BACKGROUND OF THE INVENTION

Hyponatremia is an electrolyte abnormality with a potential for significant morbidity and mortality. The condition arises when a person exhibits a positive balance of water with or without a deficit of sodium, which is recognized when the plasma sodium falls below the level of 135 mmol/L. Hyponatremia can occur in isolation in individuals who over-consume water. But more frequently hyponatremia results from a complication of medication or other underlying medical conditions leading to a diminished excretion of water.

Hyponatremia may lead to water intoxication, which results from retention of excess water causing the normal tonicity of extracellular fluid to fall below the safe limit. This may result in a potentially fatal disturbance in brain function. Typical symptoms of water intoxication include nausea, vomiting, headache, and malaise. Because nausea may also release anti-diuretic hormone (ADH), which causes water retention, this may cause a positive feedback loop that further worsens the symptoms of hyponatremia. Upon worsening conditions, a patient may experience reduced reflexes, convulsions, stupor, coma, or even death.

One way of treating hyponatremia is to administer urea to the patient. See, for example, 2014 European Society of Endocrinology, European Society of Intensive Care, European renal Association-EDTA. While the benefits of oral administration of urea are well documented, challenges exist in the development of an oral urea product that is acceptable for patients.

The inventors have perceived a need to develop urea compositions that can be packaged in a multi-use container, and resist clumping when exposed to moisture present in the environment while the product is being used.

### SUMMARY OF THE INVENTION

The invention, according to one aspect, relates to a powder composition that can be used to treat hypernatremia. The composition may comprise (a) an amount of urea effective for treatment of hyponatremia; (b) silica; and (c) calcium silicate. The calcium silicate may have a defined particle size range, such that the particle sizes are in the range of 3 to 7 microns, wherein less than 10% of the particles by weight have a diameter less than 3 microns or greater than 7 microns. The powder composition does not contain a flavoring agent that may interfere with its desirable anti-clumping properties. Accordingly, the product is resilient to intermittent exposure to environmental humidity. The compositions of the invention are useful in delivering urea to patients suffering from hyponatremia, in a form that is useful for oral administration.

In another aspect, the invention relates to a powder composition that includes (a) 95-99.5 wt % of urea; and (b) 0.5-5 wt % silica; (c) 0.05-1 wt % calcium silicate. The calcium silicate may have a defined particle size range, such that the particle sizes are in the range of 3 to 7 microns, wherein less than 10% of the particles by weight have a diameter less than 3 microns or greater than 7 microns. The powder composition does not contain a flavoring agent that may interfere with its desirable anti-clumping properties. Accordingly, the product is resilient to intermittent exposure to environmental humidity. The compositions of the invention are useful in delivering urea to patients suffering from hyponatremia, in a form that is useful for oral administration.

In another aspect, the invention relates to methods of treating hyponatremia by orally administering these urea compositions. This may involve dispersing or dissolving the product in water, followed by ingestion. Alternatively, the powder may be added to food or a pre-packaged food product.

### DETAILED DESCRIPTION OF THE INVENTION

The present invention can be broadly described as a more stable powder composition comprising an amount of urea effective for treatment of hyponatremia. It may also involve treating those suffering from hyponatremia, or who are at risk from developing hyponatremia, with the urea composition of the invention. The composition may be dispersed in water or another liquid before ingestion by the patient, or may be added to food. Alternatively, the composition may be added to food and packaged until it is ready for use.

The invention also relates to methods of making urea compositions that avoid manufacturing and packaging problems arising from the active ingredient, urea. The inventors have identified several difficulties in the manufacturing of a stable urea composition that arise due to the unique properties of urea, including its hygroscopic nature. For example, the processing of Ure-Na powder has been complicated by sticking of the powder and clumping of the product onto to various parts of the manufacturing and packaging machinery. This required frequent stopping the manufacturing line and cleaning of the processing equipment in order to maintain consistent product quality.

The inventors found a novel formulation that prevents the urea product from substantially clumping after repeated or excessive exposure of the urea product to environmental humidity. This clumping issue has been addressed and solved by adding silica, also known as silicon dioxide, to the improved formulation of Ure-Na™ as previously discussed while removing any flavoring agent. In one embodiment, the formulation comprises a urea, a calcium silicate, and the new addition of a silica. In one embodiment, the silica is SIPERNAT® 50 S which is produced by Evonik. In one embodiment, the calcium silicate is ZEOFREE® 600 which is also produced by Evonik, formerly known as Hubersorb® 600. The formulation preferably does not include any flavoring agents, which have been found to unexpectedly results in clumping when examples of the preferred embodiments containing them are subjected to repeated exposure to ambient moisture, as when the product is stored in a multi-use container.

In one embodiment, the bulk urea product has a composition preferably between 80-99.5 wt %, more preferably 95-99.5 wt %, and most preferably about 98 wt % urea. The composition preferably comprises 0.05-5 wt %, more preferably 0.05-1 wt %, and most preferably about 0.33 wt % calcium silicate. The calcium silicate typically includes 99% calcium silicate, and 1% sodium sulfate by weight. The sodium sulfate therefore may be present in the urea powder composition. In one embodiment, the calcium silicate has a mean particle diameter within the range of 3.0 and 7.0 microns, and less than 10% of the particles by weight being below 3.0 micron or above 7.0 micron in diameter. The composition preferably comprises about 0.1-10 wt %, more preferably about 0.5-5 wt %, and most preferably about 1.63 wt % silica. In one embodiment, the silica has a mean particle diameter in the range of preferably about 5-50 microns, more preferably 10-25 microns, and most preferably 15-21 microns. In one embodiment, the silica is preferably greater than 90% silicon dioxide, more preferably 95% silicon dioxide, and most preferably greater than 97% silicon dioxide.

The bulk urea product omits the presence of a flavoring agent unlike that of Ure-Na™. Multiple iterations of a bulk urea product were formulated resulting in many formulations that failed to exhibit the resilience to clumping that is needed for urea product suitable for packaging in a multi-use container. Several urea flavoring formulations were created and each one exhibited poor anticlumping properties in a bulk container. In one example, a grape flavoring agent was used in a urea formulation with the calcium silicate ZEOFREE® 600, and the silica SIPERNAT® 50 S. This formulation exhibited poor anticlumping properties. In a second example, a citrus flavoring agent was used because it requires a smaller amount of flavoring agent than that of the grape. This formulation comprised of the citrus flavoring agent, the calcium silicate ZEOFREE® 600, and the silica SIPERNAT® 50 S. This formulation additionally exhibited poor anti-clumping properties. Other flavoring agents were used in multiple different formulations which all resulted in poor anti-clumping properties.

The only successful formulation tested of the bulk urea product with the silica, SIPERNATO, was the formulation with no flavoring agent. This formulation exhibited excellent anticlumping properties. This formulation was resilient to clumping over extended periods of time at environmental humidity levels. Environmental humidity levels indoors are typically between 30% and 50% humidity. The bulk formula with this formulation even exhibited resilience to clumping when continuously exposed to environmental humidity for weeks at a time. The relationship between the silica, SIPERNAT® 50 S, and the flavoring agents is unexpected.

In one embodiment, a packaged bulk product of the urea-silica formulation above may contain 30 doses, wherein the packaged product would be expected to be opened and closed 30 times wherein each opening exposes the remaining product to the environmental humidity. In one embodiment, a dose is about a 15 g scoop of the bulk urea product. In another embodiment, the packaged product may comprise 5-500 doses, 10-100 doses, 10-50 doses, 20-30 doses, or any whole or non-whole number of doses within any of the ranges stated.

### Comparative Example 1

The present inventors described in U.S. Pat. No. 11,318,092, granted on May 3, 2022, and entitled “Nutritional Composition for the Management of Hyponatremia,” which is incorporated by reference in its entirety, a urea composition that avoids clumping when individually packaged in moisture-resistant packaging. The following ingredients were added mixer to produce a dry powder:

| | | |
|---|---|---|
| Component | Amount | wt % |

| | | | |
|---|---|---|---|
| USP Urea | 15000 | mg | 67.0% |
| Sucralose | 210 | mg | 0.9% |
| Citric acid (anhydrous) | 1600 | mg | 7.1% |
| Natural Lemon-Lime Flavor | 2300 | mg | 10.3% |
| Maltodextrine M-500 | 2170 | mg | 9.7% |
| Calcium Silicate (particle size 3-7 μm) | 1120 | mg | 5.0% |

The calcium silicate used in this example is Zeofree 600 (formerly known as Hubersorb 600). This calcium silicate is characterized as having a particle size distribution where significant amounts of calcium silicate particles below 3.0 microns, and above 7.0 microns, are avoided. Thus, it is preferred that the calcium silicate have an mean particle diameter within the range of 3.0 and 7.0 microns, and less than 10% of the particles by weight being below 3.0 micron or above 7.0 micron in diameter. In another embodiment, the calcium silicate has a mean particle diameter within the range of 4.0 and 6.0 microns, desirably a median particle size of 5.0 micron, and less than 10% of the particles by weight being below 4.0 micron and above 6.0 micron in diameter. While the threshold of 10% of particles by weight being outside the target particle diameter range is acceptable. Preferably, even lower amounts of off-target particles is desirable, such as 8%, 6%, 4%, or even 1%. For example, a composition having a mean particle diameter within the range of 4 and 6 microns, with less than 1% by weight outside of that range may be desirable.

The composition is prepared by adding the ingredients into a mixer to produce a powder product. The resulting powder is free flowing and resistant to clumping. The powder composition is preferably packaged in a pouch that can be sprinkled onto food, or mixed with a liquid such as water. The composition forms a suspension when mixed with water, which allows for convenient ingestion. In this sense, the powder can be distributed and sold as a medical food. Alternatively, the powder may be incorporated into another medical food product such as a cookie or bar form similar to a diet bar.

The product of Example 1 was packaged in pouches using the same packaging used for the previous Ure-Na™ product that lacked calcium silicate. The consumer returns associated with clumping for the previous Ure-Na™ product was 1.4%. Return data for the product of Example 1 was 0.2%. Therefore, the present invention resulted in seven-fold reduction in returns due to clumping relative to existing Ure-Na™ product.

The present inventors have observed that the product described in this example when packaged in a multi-use container exhibits clumping due to repeated exposure to ambient moisture.

### Comparative Example 2

The following ingredients were added mixer to produce a dry powder:

| | | |
|---|---|---|
| Component | Amount | wt % |

| | | | |
|---|---|---|---|
| USP Urea | 15000 | mg | 85.2% |
| Calcium Silicate (particle size 3-7 μm) | 50 | mg | 0.3% |
| Natural Lemon-Lime Flavor | 2300 | mg | 13.1% |
| Silica (Sipernat 50S) | 250 | mg | 1.4% |

This composition exhibited clumping when the product packaged in a multi-use container.

### Comparative Example 3

The following ingredients were added mixer to produce a dry powder:

| | | |
|---|---|---|
| Component | Amount | wt % |

| | | | |
|---|---|---|---|
| USP Urea | 15000 | mg | 80.8% |
| Calcium Silicate (particle size 3-7 μm) | 50 | mg | 0.3% |
| Grape Flavor | 3000 | mg | 16.2% |
| Prinova (bitter blocker) | 275 | mg | 1.5 |

| Component | Amount | wt % |
|---|---|---|
| Silica (Sipernat 50S) | 250 mg | 1.3% |

This composition exhibited clumping when the product packaged in a multi-use container.

### Example 4

The following ingredients were added mixer to produce a dry powder:

| | | |
|---|---|---|
| Component | Amount | wt % |

| | | | |
|---|---|---|---|
| USP Urea | 15000 | mg | 98.0% |
| Calcium Silicate (particle size 3-7 μm) | 50 | mg | 0.3% |
| Silica (Sipernat 50S) | 250 | mg | 1.6% |

This composition unexpectedly exhibited complete stability (no clumping observed over a period of several months) after repeated exposure to ambient moisture. This urea composition was determined to be suitable for multi-use packaging due to the lack of clumping being observed after repeated exposure to ambient moisture.

Other embodiments and uses of the invention will be apparent to those skilled in the art from consideration of the specification and practice of the invention disclosed herein. All references cited herein, including all U.S. and foreign patents and patent applications, are specifically and entirely hereby incorporated herein by reference. It is intended that the specification and examples be considered exemplary only, with the true scope and spirit of the invention indicated by the following claims.

What is claimed is:

1. A urea composition comprising:
(a) an amount of urea effective for treatment of hyponatremia;
(b) silica; and
(c) calcium silicate.

2. The composition of claim 1, wherein the composition does not clump when exposed to indoor humidity levels of about 40% for 24 hours.

3. The composition of claim 1, wherein the composition does not clump when exposed to indoor humidity levels of about 50% for 24 hours.

4. The composition of claim 1, the calcium silicate having a particle size in the range of 3 to 7 microns, wherein less than 10% of the particles by weight have a diameter less than 3 microns or greater than 7 microns

5. The composition of claim 1, the silica having a mean particle size in the range of 10-25 microns.

6. The composition of claim 1, wherein the composition comprises 95-99.5 wt % urea.

7. The composition of claim 1, wherein the composition comprises 0.05-1 wt % calcium silicate.

8. The composition of claim 1, wherein the composition comprises 0.5-5 wt % silica.

9. The composition of claim 1, wherein the composition lacks a flavoring agent.

10. A method of treating hyponatremia comprising administering the composition of claim 1 to a patient suffering from hyponatremia.

11. The method of claim 10, wherein the composition is administered by dispersing or dissolving the composition in water prior to consumption.

12. The method of claim 10, wherein the composition is administered by mixing the composition with food prior to consumption.

13. A powder composition comprising:
(a) 95-99.5 wt % of urea;
(b) 0.5-5 wt % silica; and
(c) 0.05-1 wt % calcium silicate.

14. The powder composition of claim 13, the calcium silicate having a particle size in the range of 3 to 7 microns, wherein less than 10% of the particles by weight have a diameter less than 3 microns or greater than 7 microns.

15. The powder composition of claim 13, the silica having a mean particle size in the range of 10-25 microns.

16. The powder composition of claim 13, wherein the composition does not clump when exposed to indoor humidity levels of about 40% for 24 hours.

17. The powder composition of claim 13, wherein the composition does not clump when exposed to indoor humidity levels of about 50% for 24 hours.

18. The powder composition of claim 13, wherein the composition lacks a flavoring agent.

19. A method of treating hyponatremia comprising administering the composition of claim 13 to a patient suffering from hyponatremia.

20. The method of claim 18, wherein the composition is administered by dispersing or dissolving the composition in water prior to consumption.
